(19) United States
(12) Patent Application Publication
(10) Pub. No.: US 2024/0016453 A1
(43) Pub. Date: Jan. 18, 2024

(54) WIRELESS IMPLANTABLE DEVICE AND METHOD FOR MONITORING INTERNAL PHYSIOLOGICAL PARAMETERS OF ANIMALS

(71) Applicants: PURDUE RESEARCH FOUNDATION, West Lafayette, IN (US); The United States of America, as Represented by the Secretary of Agriculture, Washington, DC (US)

(72) Inventors: Tyler C. Field, West Lafayette, IN (US); Samantha Marie Neeno, West Lafayette, IN (US); Robert Merton Stwalley, III, Lafayette, IN (US); Allan P. Schinkel, West Lafayette, IN (US); Jay S. Johnson, West Lafayette, IN (US)

(21) Appl. No.: 18/351,037

(22) Filed: Jul. 12, 2023

Related U.S. Application Data
(60) Provisional application No. 63/368,207, filed on Jul. 12, 2022.

(51) Int. Cl. A61B 5/00; A61B 5/01; A61B 7/02; A61B 5/1455

(52) U.S. Cl. CPC A61B 5/6867; A61B 5/01; A61B 5/6882; A61B 7/023; A61B 5/14552; A61B 5/0031; A61B 2560/0219; A61B 2503/40

(57) ABSTRACT

Devices and methods for monitoring internal physiological parameters of animals. Such a device and method makes use of a housing configured for implantation in an animal, and at least first and second arms pivotally coupled to the housing to have a collapsed configuration and a deployed configuration, wherein the first and second arms are alongside the housing and approximately parallel to the longitudinal axis in the collapsed configuration, expanded outward away from the housing and not parallel to the longitudinal axis in the deployed configuration, and biased toward the deployed configuration. Sensors are associated with the housing for collecting physiological parameters of the animal. The device is further equipped with a wireless transmitter for wirelessly transmitting outputs of the sensors to a receiver while the housing is implanted internally within the animal and the receiver is located externally of the animal.

### CROSS-REFERENCE TO RELATED APPLICATIONS

This application claims the benefit of U.S. Provisional Application No. 63/368,207 filed Jul. 12, 2022, the contents of which are incorporated herein by reference.

### BACKGROUND OF THE INVENTION

The present invention generally relates to devices and methods for monitoring physiological parameters of living bodies. The invention particularly relates to a wireless implantable device and method for monitoring internal physiological parameters of animals.

Modern domestic animal production, particularly hog production, is generally limited by the amount of thermal stress that an animal can reasonably tolerate. As a nonlimiting example, skin temperatures between sows under similar heat stress conditions can vary greatly. While respiration rates and rectal temperatures have a stronger correlation, they require significantly more manpower to measure and record for research studies, and they are impractical to collect for large commercial operations. Current devices for measuring the internal temperature of an animal are either passive devices that require personnel to measure each animal individually by applying a wireless power source momentarily to the skin or are battery-powered devices that record the temperature at regular intervals, but must be retrieved before the data can be accessed and analyzed.

It would be desirable if improved devices and methods were available for addressing the shortcomings of current devices utilized to measure internal temperatures and other physiological parameters of animals.

### BRIEF SUMMARY OF THE INVENTION

The intent of this section of the specification is to briefly indicate the nature and substance of the invention, as opposed to an exhaustive statement of all subject matter and aspects of the invention. Therefore, while this section is intended to be directed to and consistent with certain subject matter recited in the claims, additional subject matter and aspects relating to the invention are set forth in other sections of the specification, particularly the detailed description, as well as any drawings.

The present invention provides, but is not limited to, devices and methods for monitoring internal physiological parameters of animals.

According to a nonlimiting aspect of the invention, a wireless implantable device for monitoring internal physiological parameters of an animal includes a housing having a cylindrical external shape, a longitudinal axis, and an internal compartment, wherein the housing is configured for implantation in the animal. The device further includes at least first and second arms pivotally coupled to the housing to have a collapsed configuration and a deployed configuration, wherein the first and second arms are alongside the housing and approximately parallel to the longitudinal axis in the collapsed configuration, expanded outward away from the housing and not parallel to the longitudinal axis in the deployed configuration, and biased toward the deployed configuration. Sensors, including but not limited to a temperature sensor and an acoustic sensor, are associated with the housing for collecting physiological parameters of the animal. The device is further equipped with a wireless transmitter for wirelessly transmitting outputs of the sensors to a receiver while the housing is implanted internally within the animal and the receiver is located externally of the animal.

Another nonlimiting aspect of the invention is a method of using the device comprising the elements described above.

Technical aspects of devices and methods having features as described above preferably include the capability of monitoring physiological parameters, for example, deep body temperatures, of an animal and automatically transmitting data relating thereto at regular intervals to an external remote device, for example, a central data computer, so that real time decision making can occur with respect to the care of the animal With this device, large-scale data collection is feasible with a modest-sized staff.

Other aspects and advantages will be appreciated from the following detailed description.

### BRIEF DESCRIPTION OF THE SEVERAL VIEWS OF THE DRAWING

FIG. 1 schematically represents an anatomical region of a sow and the placement of a wireless implantable device within the vagina of the sow in accordance with a nonlimiting aspect of this invention.

FIGS. 2 and 3 are schematic representations of the device of FIG. 1, in which the device is shown in deployed and stowed configurations, respectively.

FIG. 4 is another schematic representation of the device of FIG. 1.

FIG. 5 is a schematic representation of the device of FIG. 1, in which an interior of the device is exposed.

FIG. 6 is an image indicating a measured steady-state thermal cross-section of the device of FIG. 1 while implanted in the vagina of a sow.

FIG. 7 is a graph plotting the received signal strength of the device of FIG. 1 with and without interference caused by pork flesh.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of what is depicted in the drawings. The following detailed description also describes certain investigations relating to the embodiment(s), and identifies certain but not all alternatives of the embodiment(s). As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

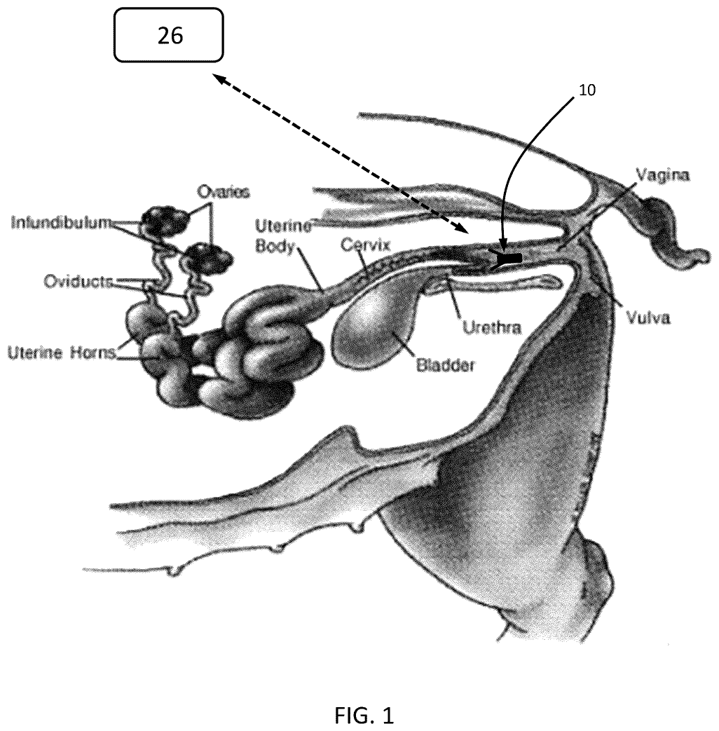

FIGS. 1 through 5 schematically represent a nonlimiting embodiment of a wireless implantable device 10 and FIG. 1 depicts the device implanted in the vagina of a sow. As a matter of convenience, the device and methods of using the device will be illustrated and described hereinafter in reference to implantation in the vagina of a sow. However, it will be appreciated that the teachings of the invention may also be generally applicable to other animals and methods and locations for implantation.

Sow heat stress is multifaceted, and the prompt mitigation of the condition can have a significant positive material impact on production economics. As sows are exposed to temperatures outside their thermoneutral zone, they must utilize their own energy to maintain their internal body temperature. Lactating sows are particularly susceptible to thermal stress due to the heat generation of milk production. Because pigs do not sweat, heat stress is typically remedied by reducing high-energy bodily functions, such as milk production. This lower level of milk production impacts piglets in several ways, including a delayed wean to estrus interval, lower body condition score at weaning, and lower piglet quality. External cooling reduces the signs of heat stress in sows and piglets. As sows exceed the evaporative critical temperature, their skin temperature, respiration rate, and internal body temperatures increase, in this approximate order. Skin and rectal temperatures are used to approximate internal body temperature, and the differential between these values is indicative of the heat stress severity.

Existing devices and methods of detecting individual sow heat stress do not permit continuous real-time monitoring that would enable prompt and effective interventions by farm personnel. Continuous monitoring options include subcutaneous, digestive, and vaginal insertion of thermal sensors. These locations are more closely correlated to the internal body temperature than typical skin temperature monitoring. Skin temperature data can have low signal-to-noise ratios, due to the skin's exposure to environmental variables. Unfortunately, its usefulness as a correlative variable for heat stress is dependent on where and how measurements are collected on the body such as by an infrared (IR) gun or camera. Caution must be used by potential researchers, and they need to understand that variability in skin temperatures can result from the manner in which the reading is obtained and where on the animal's body it is measured. Although the continuous collection of vaginal temperature data from button sensors exists, these devices are typically left in the sow for the duration of lactation, with data stored onboard the device 10, to be collected at the trial's end. This methodology can be improved by incorporating wireless transmission of data, with continuous real-time monitoring and treatment intervention becoming possible.

The following describes provides a description of a particular but nonlimiting embodiment of the wireless implantable device 10 with capabilities that address the above drawbacks of existing devices and methods used to detect individual sow heat stress. Additionally, the device 10 provides for the ability to monitor physiological parameters in addition to temperature.

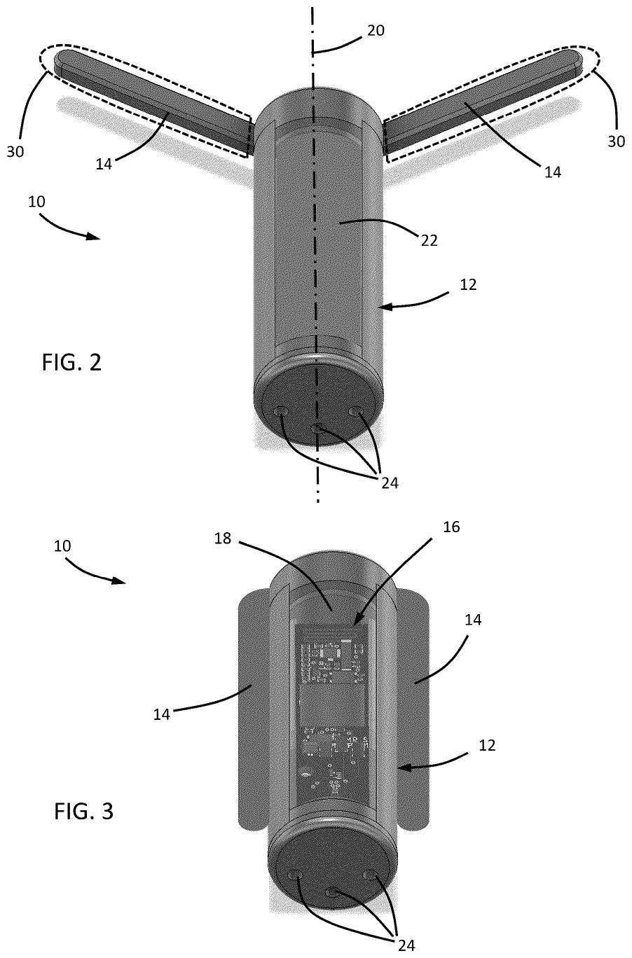

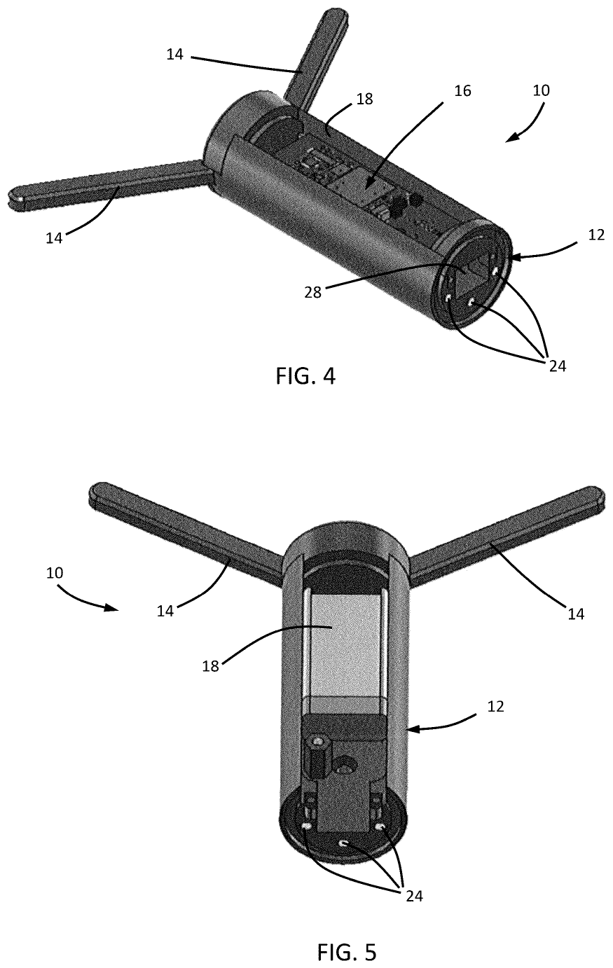

The device 10 is represented in FIGS. 1 through 5 as including a housing 12, first and second arms 14, and electronic circuitry 16 disposed within an internal compartment 18 within the housing 12 (FIGS. 3 and 4). While only two arms 14 may be suitable or preferred for some embodiments, it is foreseeable that devices 10 equipped with more than two arms 14 could be desirable and such embodiments are within the scope of the invention. The housing 12 is represented as having a cylindrical external shape with a longitudinal axis 20, and the compartment 18 is represented as accessed through an opening that is closable with a removal panel 22. The external shape of the housing 12 is generally configured to facilitate placement (implantation) of the device 10 in the vagina of a sow. The first and second arms 14 are pivotally coupled to the housing 12 to have a collapsed configuration (FIG. 3) and a deployed configuration (FIGS. 1, 2, 4, and 5), the latter configuration being adapted to retain the device 10 within the vagina of a sow. As evident from comparing FIGS. 3 and 4, the arms 14 can be pivotally coupled to the housing 12 so as to be alongside the housing 12 and approximately parallel to the longitudinal axis 20 in the collapsed configuration (FIG. 3), and expanded outward away from the housing 12 and not parallel to the longitudinal axis 20 in the deployed configuration (FIG. 2). In the embodiment shown, each of the arms 14 is oriented at an acute angle to the longitudinal axis 20 of the housing 12 when positioned in their respective deployed configurations. Though the arms 14 are represented as straight (rectilinear) in the drawings, it is foreseeable that arms 14 of different shapes could be utilized.

The arms 14 are preferably biased toward the deployed configuration. For example, the arms 14 can be pivotally coupled to the housing 12 with the assistance of torsion springs (not shown) that serve as biasing means. To reduce the risk of damage to surrounding tissue, the biasing means preferably biases the arms 14 toward the deployed configuration to generate a force on each arm 14 of about 2 to about 3 ounces, which is believed to be capable of generating sufficient pressure to retain the device 10 within the vagina of a sow. The housing 12 is preferably equipped with a latch or other means for securing the arms 14 in the stowed configuration, and a release button or other means for disengaging the latch to release the arms 14 from the stowed configuration. In the deployed configuration, the device 10 is generally Y-shaped, with all of the electronics housed in the central housing 12 of the device 10. The arms 14 are preferably interchangeable with arms of different lengths to vary the retaining pressure applied by the arms 14 to the animal's vagina, which should accommodate the general increase in internal diameter of sows from their increasing parity.

FIG. 2 schematically represents each arm 14 as optionally encased in a pliable and elastic enclosure 30. The enclosures 30 may be formed partially or entirely of a silicone rubber or another material that is biocompatible to enable the device 10 to remain implanted for extended durations. The enclosures 30 may be attached to their respective arms 14 in any suitable manner and are preferably capable of being inflated or otherwise expanded to increase the effective lengths and/or cross-sections of the arms 14 that they encase, with the intent of increasing the effectiveness of each arm 14 to retain the device 10 within the vagina of a sow. The relative size of an enclosure 30 to its arm can vary widely from that represented in FIG. 2, for example, to tailor the retention capability contributed by the enclosure 30.

Sensors 24 are schematically represented as associated with the housing 12, in this example, disposed in a wall of the housing 12 at one end thereof to enable the sensors 24 to be located in close proximity to the cervix when the device 10 is placed in the vagina. Placement of the temperature sensor proximal to the sow's cervix is desirable to minimize temperature variation from the environment and ensure the sensed vaginal temperature approximates the true core temperature of the animal. The sensors 24 are connected to the circuitry 16 by which the sensors 24 can be controlled and outputs of the sensors 24 optionally processed before being wireless transmitted to an external receiver 26 positioned externally of the animal (FIG. 1). The sensors 24 are adapted to collect physiological parameters of the animal of interest, nonlimiting examples of which include temperature sensors, acoustic sensors, optical sensors, and viscosity sensors. According to preferred but nonlimiting aspects of the invention, a temperature sensor utilized by the device 10 is preferably configured to sense a deep body temperature of the animal. An acoustic sensor utilized by the device 10 is configured to sense deep body acoustics of the animal, for example, for the purpose of sensing at least one of the pulse, respiration rate, blood pressure, and digestive tract activity of the animal. An optical sensor utilized by the device 10 is preferably equipped with a light emitter and a light receiver, wherein the light sensor is configured to collect light data generated by light emitted by the light emitter for use in determining at least blood oxygen content of the animal. A viscosity sensor utilized by the device 10 is preferably configured to collect viscosity data of vaginal fluid of the animal, as an example, for use in determining at least the onset of estrus of the animal.

For purposes of transmitting the outputs of the sensors 24 to the external receiver 26, the circuitry 16 preferably includes a radio transmitter or other suitable wireless transmitting device. For purposes of interacting with the sensors 24 and their respective outputs, the circuitry 16 may include edge computing circuitry capable of at least preliminarily processing the outputs of one or more of the sensors 24, and means for data buffering the outputs of one or more of the sensors 24. The compartment 18 may also contain a power source (such as a battery) for supplying electrical power to the sensors 24 and the wireless transmitting means, in which case the housing 12 can be equipped with a USB port 28 (FIG. 4) or other suitable connection for charging the power source.

The housing 12 and arms 14 of the device 10 are preferably constructed of appropriate exterior materials to ensure biocompatibility with the contact interface of tissue, which in the case represented in FIG. 1 is the tissue within the vagina of a sow. Suitable materials include but are not limited to polycarbonate and stainless steels for structural components and silicone or other biocompatible materials for sealing the housing 12 around its internal compartment 18.

In investigations leading to the present invention, an experimental device 10 generally as represented in FIGS. 2 through 5 was fabricated and equipped with the electronic circuitry 16 that included was a microcontroller and a 915 MHZ LoRa radio as the wireless transmitter. The radio was chosen over WiFi wireless transmitters primarily for its ability to transmit without needing a handshake connection to a specific receiver, enabling any device that is listening to receive the transmitted data and eliminating the passive power required to simply maintain a handshake, even if no data are being transmitted. Additionally, the 915 MHZ frequency has a longer wavelength than frequencies used for WiFi, which allowed better transmission through physical obstructions. The microcontroller was powered by a rechargeable lithium-ion battery and had an onboard power regulator that limited the current draw to 500 mA at 3.3 VDC. These devices could then communicate with a single central LoRa receiver microcontroller that would log time-stamped temperature data upon reception.

A computational heat conduction analysis was performed with the assumption that 100% of the peak power of 1.65 W was constantly being rejected as heat. This allowed for the determination of an upper bound for the temperature at the interface between the experimental device 10 and a sow into which the device 10 would be implanted to ensure that any heat generated by the device 10 could be adequately dispersed and removed from the animal, rather than accumulating near the vagina and subsequently raising its temperature and causing discomfort to the animal. For the investigations, the vagina of a sow was approximated as a cylindrical body 0.6 m in diameter and 1.5 m long. The experimental device 10 was modeled as a cylinder 3 cm in diameter and 13 cm long, with the rear of the device 10 located 10 cm into the interior of the rear end of the sow. The external surfaces of the sow were set to convective boundary conditions, with an ambient temperature of about 35° C. to minimize the sow's ability to reject heat to the environment. The sow's heat generation was not included in the analysis to better visualize the heating solely due to the device 10.

To test the device 10 and verify that the 915 MHZ signal could be received through a sow at varying transmission distances, a variety of cuts of pork were acquired from a local butcher and stacked on a wheeled cart to simulate a sow. The device was then inserted into the approximate center of the pork, and the cart was moved to multiple distances from the receiving system. Received signal strength was then determined for each distance. This protocol was repeated in triplicate and for multiple transmission powers to determine the best settings for future live animal tests. Once a transmission power level was selected, the device 10 was set to transmit the voltage of the battery once each minute until failure. The rate of voltage decrease was then used to verify the current draw specifications provided on the manufacturer's website and create a predictive model for battery life at different transmission powers and transmission frequencies, with the goal of proper battery selection to ensure reliable data transmission for the entirety of a twenty-one day lactation as it was deemed of importance for the device 10 to operate for the duration of sow lactation without human intervention. Two transmission protocols were examined for their ability to provide this operational period on a single battery charge.

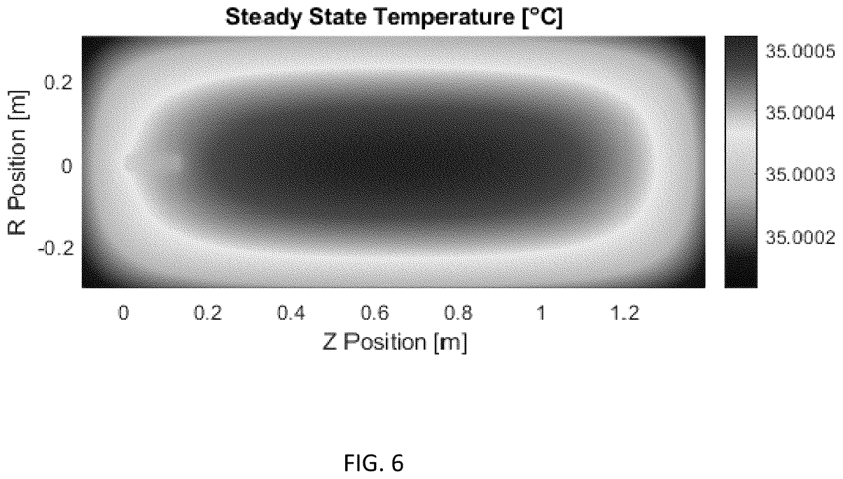

Without the heat generation from the device 10, the steady state temperature of the sow would be uniform and equivalent to the ambient air temperature of 35° C. Therefore, any variation from this ambient temperature in the model with the device 10, would be due to the electrical heat dissipation of the device 10. FIG. 6 shows a cross sectional view of the device 10 and simulated sow vagina with the rear end of the device 10 at the origin. The device 10 can be identified due to the slightly elevated temperature difference, but the temperature range over the entire domain is only 0.0004° C. and just 0.0005° C. greater than the ambient temperature. This demonstrated that even at the upper bound of possible heat generation from the device 10, the temperature of a sow would be only negligibly increased, compared to a sow with no device 10. The device 10 should therefore be safe and non-discomforting for a sow from a thermal perspective.

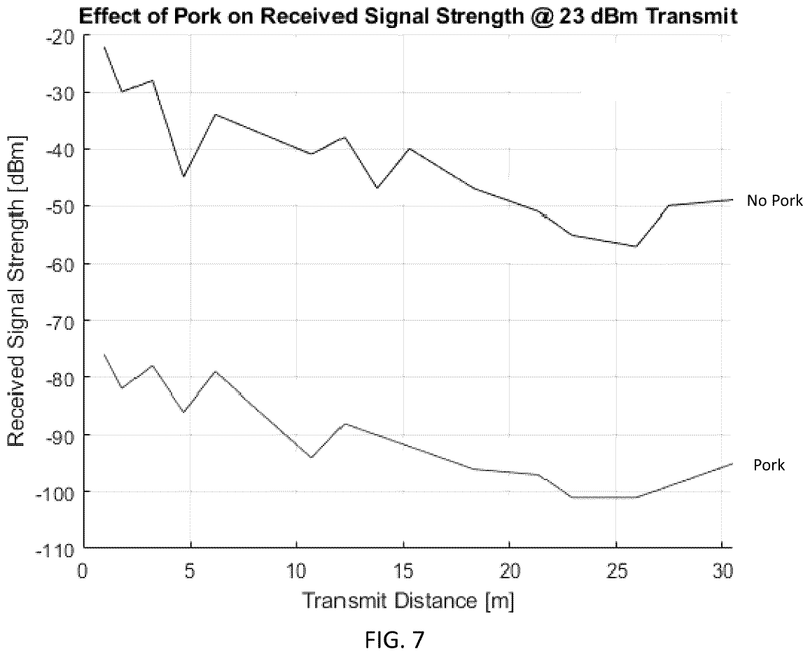

The signal strength received from the device 10 was tested at fifteen longitudinal distances, ranging from 0 m to 30 m with a 1 m lateral offset. The cart with the device 10 was rolled down a long hallway, and the receiver 26 was kept stationary at one end. Three measurements were taken at each distance and then averaged. This was then repeated with the pork on the cart and the device 10 at a depth of approximately 13 cm within the pork. Under these conditions and at a transmission power of 23 dBm, the pork reduced the received signal strength by 48±4 dBm on average. There was a measurable drop-off in signal strength with distance, but reliable transmission occurred throughout all the distances tested as seen in FIG. 7.

The signal strength with the pork was also measured with transmission powers of 10 and 16 dBm. It was expected that the received signal strength would be reduced for lower transmit powers, but at the majority of distances, the greatest received signal strength was with a transmission power of 16 dBm. As a result, a negative quadratic fit to the signal strengths at each of these distances was used to identify the optimal transmission power and to maximize the received signal strength. The optimal transmission power was determined to be 16.4±1.3 dBm. When this study was later repeated with a sacrificed male pig, an incision was made in the abdomen, and the sensor was fully inserted into the body cavity. The received signal strengths measured during this later study showed good agreement with the pork cart experiment signal strengths, indicating that further testing could be completing using various cuts of pork as a suitable substitute for an animal.

A transmit power of 17 dBm was selected to test the life of the lithium-ion battery utilized in the experimental device 10. It was assumed that during one cycle of the device 10 that there would be a significant amount of idle time, followed by a higher current draw period, when the temperature sensor was read and the measured value transmitted. The amount of the cycle dedicated to the high current read and transmit period was determined by having the microcontroller transmit repeatedly and using the time between successive measurements as the transmission time. Transmission time was found to be approximately 780 ms. A cycle time of 1 min was then used to accelerate the discharge of the battery, compared to a more realistic cycle time of 10-15 min, but still have a relatively long idle time compared to the transmission time. The battery voltage started at almost 4.2 V and decayed relatively linearly (R2=0.9475) until approximately 3.4 V, after which it rapidly decayed down to 3.0 V, before failing to transmit further. A potential of 3.4 V was selected as the voltage where the battery would be considered fully discharged, in order to conservatively estimate the battery life and allow for a factor of safety in the design of the final device 10.

The average current draw of the experimental device 10 was identified using two methods. The first technique incorporated the average value of the linear fit, while the second utilized the stated energy content of the battery and the time it took to discharge. These two methods showed good agreement in the average current draw over the whole projected cycle of 13.7 mA and 13.8 mA, respectively. The idle current of the selected microcontroller was specified as approximately 11.5 mA. This value was then used along with the idle and active cycle times to work out a current draw period during the active part of the cycle of approximately 181 mA.

A predictive model was developed using this data to estimate battery life with varying cycle times and transmit powers, as well as considering the use of the Adafruit SleepyDog library to provide a low power sleep mode with a minimal 3 mA current draw. Longer cycle times and lower transmission powers increase the time it takes for the battery to discharge. The model assumed a linear voltage decrease with each cycle, and it had an error of less than one hour when compared with the above test case. With the implementation of the SleepyDog library in the final version of the software, the predicted battery life with a 2000 mAh battery, twelve-minute cycle time, and 17 dBm transmit power was twenty-six days and two hours, which would allow for the device to be inserted soon after farrowing and remain in place for the duration of a twenty-one-day lactation. This would provide a reasonable buffer for battery decay from repeated charge/discharge cycles over the life of the device 10. It was also seen that the effect of cycle time on battery life was much more significant than that of transmission power, although a small change in transmission power is more significant near the maximum power level of the radio.

On the basis of the above investigations, it was concluded that the device 10 was capable of continuously delivering biometric data in real-time via a wireless transmitter and onboard battery. The thermal model evidenced that excess heat would not be retained by an animal in which the device 10 was implanted. The maximum temperature rise within the sow simulation was shown to be less than 0.001° C., even with all electrical power being continually rejected as heat. Lastly, a predictive model was developed to estimate the discharge time of the lithium-ion battery at different transmission powers and cycle times, as well as with and without additional software libraries that can further reduce power consumption between data transmissions.

While the investigations were directed to a device 10 that incorporated a temperature sensor, other physiological parameters, including but not limited to heart rate and respiration rate, are believed to be capable of being sensed and monitored by incorporating an acoustic device, such as a small microphone, on the housing 12 of the device 10. By measuring these parameters internally, the amount of external noise would be dampened by the body of the animal and the likelihood of the device 10 becoming dislodged would be decreased. Similarly, the aforementioned optical and/or viscosity sensors can be incorporated on the housing 12 of the device 10.

As previously noted above, though the foregoing detailed description describes certain aspects of one or more particular embodiments of the invention and investigations associated with the invention, alternatives could be adopted by one skilled in the art. For example, the device 10 and its components could differ in appearance and construction from the embodiment described herein and shown in the drawings, as a nonlimiting example, by the inclusion of additional arms 14 and/or arms 14 that are not linear in shape. In addition, functions of certain components of the device 10 could be performed by components of different construction but capable of a similar (though not necessarily equivalent) function, and appropriate materials could be substituted for those noted. As such, and again as was previously noted, it should be understood that the invention is not necessarily limited to any particular embodiment described herein or illustrated in the drawings.

1. A wireless implantable device for monitoring internal physiological parameters of an animal, the device comprising:
a housing comprising a cylindrical external shape, a longitudinal axis, and an internal compartment, the housing being configured for implantation in the animal;
at least first and second arms pivotally coupled to the housing to have a collapsed configuration and a deployed configuration, the first and second arms being alongside the housing and approximately parallel to the longitudinal axis in the collapsed configuration, expanded outward away from the housing and not parallel to the longitudinal axis in the deployed configuration, and biased toward the deployed configuration;
sensors associated the housing for collecting physiological parameters of the animal, the sensors comprising at least a temperature sensor and an acoustic sensor; and
wireless transmitting means within the compartment for wirelessly transmitting outputs of the sensors to a receiver while the housing is implanted internally within the animal and the receiver is located externally of the animal.

2. The wireless implantable device according to claim 1, wherein the temperature sensor is configured to sense a deep body temperature of the animal and the acoustic sensor is configured to sense deep body acoustics of the animal.

3. The wireless implantable device according to claim 2, wherein the acoustic sensor is configured to sense at least one of the pulse, respiration rate, blood pressure, and digestive tract activity of the animal.

4. The wireless implantable device according to claim 1, further comprising an optical sensor associated with the housing that includes a light emitter and a light receiver, the light sensor being configured to collect light data generated by light emitted by the light emitter for use in determining at least blood oxygen content of the animal.

5. The wireless implantable device according to claim 1, further comprising a viscosity sensor configured to collect viscosity data of vaginal fluid of the animal for use in determining at least the onset of estrus of the animal.

6. The wireless implantable device according to claim 1, wherein in the deployed configuration each of the first and second arms is oriented at an acute angle to the longitudinal axis of the housing.

7. The wireless implantable device according to claim 1, further comprising:
means for securing the first and second arms in the stowed configuration; and
means for disengaging the securing means and releasing the first and second arms from the stowed configuration.

8. The wireless implantable device according to claim 1, further comprising means for biasing the first and second arms toward the deployed configuration, the biasing means generating a force on each of the first and second arms of about 2 to about 3 ounces.

9. The wireless implantable device according to claim 1, further comprising at least one elastic enclosure encasing the first arm.

10. The wireless implantable device according to claim 9, wherein the enclosure is inflatable to increase an effective length and/or cross-section of the first arm encased by the enclosure.

11. The wireless implantable device according to claim 1, further comprising edge computing circuitry within the compartment that at least preliminarily processes the outputs of at least one of the sensors.

12. The wireless implantable device according to claim 1, further comprising means for data buffering the outputs of at least one of the sensors.

13. The wireless implantable device according to claim 1, further comprising a power source within the compartment and supplying electrical power to the sensors and the wireless transmitting means.

14. The wireless implantable device according to claim 13, further comprising a USB port disposed on the housing for charging the power source.

15. The wireless implantable device according to claim 1, wherein the device is sized and configured for placement in the vagina of a domestic animal.

16. The wireless implantable device according to claim 15, wherein the domestic animal is a porcine sow.

17. A method of using the wireless implantable device according to claim 1, the method comprising implanting the device in the vagina of a domestic animal.

18. The method according to claim 17, wherein the domestic animal is a porcine sow.

19. The method according to claim 17, further comprising wirelessly transmitting the outputs of the sensors to the receiver while the housing is implanted internally within the animal and the receiver is located externally of the animal.

20. The method according to claim 17, wherein the first and second arms are in the stowed configuration during the implanting the device, and the first and second arms are expanded to the deployed configuration after the device is implanted internally within the animal.
